(19) United States
(12) Patent Application Publication
(10) Pub. No.: US 2021/0252102 A1
(43) Pub. Date: Aug. 19, 2021

(54) METHODS FOR ENHANCING IMMUNOTHERAPY IN THE TREATMENT OF CANCER

(71) Applicants: The Texas A&M University System, College Station, TX (US); The Regents of the University of Colorado, a Body Corporate, Denver, CO (US)

(72) Inventors: Richard Tobin, Aurora, CO (US); Martha Karen Newell-Rogers, Elizabeth, CO (US)

(73) Assignees: The Texas A&M University System, College Station, TX (US); The Regents of the University of Colorado, a Body Corporate, Denver, CO (US)

(21) Appl. No.: 17/244,883

(22) Filed: Apr. 29, 2021

Related U.S. Application Data
(63) Continuation of application No. 16/646,391, filed on Mar. 11, 2020, now abandoned.
(60) Provisional application No. 62/559,499, filed on Sep. 15, 2017.
(60) Provisional application No. 62/678,214, filed on May 30, 2018.

(51) Int. Cl. A61K 38/08; A61P 35/00; C07K 16/28; G01N 33/53; G01N 33/574

(52) U.S. Cl. CPC A61K 38/08; A61P 35/00; A61K 38/1774; G01N 33/5308; G01N 33/57492; C07K 16/2818

(57) ABSTRACT

The invention relates to methods for treating cancers by targeting the elimination of selective activated immune cell populations in combination with checkpoint inhibitors. The cells may be targeted, for instance, using CLIP inhibitors and or MHC class II inducers.

### RELATED APPLICATIONS

This application claims the benefit under 35 U.S.C. § 119(e) of U.S. provisional application No. 62/559,499, filed Sep. 15, 2017, and U.S. provisional application No. 62/678,214, filed May 30, 2018, which are both incorporated by reference herein in their entireties.

### BACKGROUND OF INVENTION

Cancer remains one of the leading causes of death world-wide and the discovery of new, effective cancer treatment therapies is a critical unmet need. One of the most promising new strategies in cancer treatment has been the development of therapies that target the body's own immune system, or its components, to fight the disease. Promising approaches in this field of immunotherapy include antibodies against cancer cell components, therapeutic cancer vaccines, whole cell therapies and, most recently, drugs that target “immune checkpoint” molecules.

This latter group of immunotherapy drugs which target immune checkpoint molecules is designed to stimulate wanted or inactivate unwanted immune responses against cancer cells. Well known immune checkpoint inhibitor (“ICI”) drugs include Yervoy™, a blockade for the molecule CTLA-4, and Opdivo™ and Keytuda™, drugs designed to block the unwanted PD1:PDL1 interaction. A common feature of these ICI drug therapies is their inactivation of unwanted immune cells that seem to protect the cancer tumor from an effective anti-tumor response.

The results of cancer treatments using ICIs have been both impressive, and disappointing. For example, cancer-free survival rates for patients with advanced melanoma treated with ICI drugs are in the range of 30% to 40%. Although a positive outcome for some melanoma patients, the question remains why a large percentage of melanoma patients, roughly 60% to 70%, do not respond favorably to otherwise blockbuster ICI drugs.

### SUMMARY OF INVENTION

The invention in some aspects is a method of treating a subject having a cancer by enhancing immuno-susceptibility to therapy with checkpoint inhibitors and other therapeutics. A method of treating a subject having cancer is provided. The method involves administering to the subject an isolated MHC class II specific CLIP inhibitor and a checkpoint inhibitor.

In some embodiments the subject has a melanoma.

In some embodiments the CLIP inhibitor is administered to the subject systemically.

In other embodiments the subject is treated with the checkpoint inhibitor within 8 hours, within 24 hours, within 1 week, within 1 month or within 6 months of the CLIP inhibitor.

In some embodiments the subject is administered at least 2, 3, 4, 5, 6, 7, 8, 9, or 10 doses of CLIP inhibitor.

In some embodiments the CLIP inhibitor is administered on a regular basis to the subject. In other embodiments the CLIP inhibitor is administered to the subject daily. In yet other embodiments the CLIP inhibitor is administered to the subject every other day. In yet other embodiments the CLIP inhibitor administered to the subject weekly.

In other embodiments the subject is treated with the checkpoint inhibitor within 8 hours, within 24 hours, within 1 week, within 1 month or within 6 months of the checkpoint inhibitor.

In some embodiments the subject is administered at least 2, 3, 4, 5, 6, 7, 8, 9, or 10 doses of checkpoint inhibitor.

In some embodiments the checkpoint inhibitor is administered on a regular basis to the subject. In other embodiments the checkpoint inhibitor is administered to the subject daily. In yet other embodiments the checkpoint inhibitor is administered to the subject every other day. In yet other embodiments the checkpoint inhibitor administered to the subject weekly.

In some embodiments the checkpoint inhibitor is an antibody. In other embodiments the checkpoint inhibitor is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, and a combination thereof. In other embodiments the checkpoint inhibitor is an anti-PD-L1 antibody selected from atezolizumab, avelumab, or durvalumab. In yet other embodiments the checkpoint inhibitor is an anti-CTLA-4 antibody selected from tremelimumab or ipilimumab. In other embodiments the checkpoint inhibitor is an anti-PD1 antibody selected from nivolumab or pembrolizumab.

In some embodiments the methods are achieved by administering to the subject an isolated MHC class II specific CLIP inhibitor. The CLIP inhibitor in some embodiments is a synthetic peptide. In yet other embodiments the CLIP inhibitor is an siRNA.

In yet other embodiments the CLIP inhibitor is administered on a regular basis to the subject. For instance the CLIP inhibitor may be administered to the subject daily, every other day, or weekly.

In some embodiments the CLIP inhibitor is synthetic. In other embodiments the CLIP inhibitor is a peptide, an siRNA, or an MHC class II CLIP inhibitor. In yet other embodiments the CLIP inhibitor comprises a peptide having the sequence: X₁RX₂X₃X₄X₅LX₆X₇ (SEQ ID NO: 3), wherein each X is an amino acid, wherein R is Arginine, L is Leucine and wherein at least one of X2 and X3 is Methionine, and wherein the peptide is a CLIP displacer. The peptide in some embodiments has any one or more of the following variables: X₁ is Phenylalanine; X₂ is Isoleucine; X₃ is Methionine; X₄ is Alanine; X₅ is Valine; X₆ is Alanine; and/or X₇ is Serine.

The peptide in some embodiments includes 1-5 amino acids at the N and/or C terminus. For instance, the peptide may have 1-5 amino acid at the C terminus of X₁RX₂X₃X₄X₅LX₆X₇ (SEQ ID NO: 3) and/or the peptide may have 1-5 amino acids at the N terminus of X₁RX₂X₃X₄X₅LX₆X₇ (SEQ ID NO: 3).

The peptide in other embodiments comprises FRIM X₄VLX₆S (SEQ ID NO: 6), wherein X₄ and X₆ are any amino acid. Optionally X₄ and X₆ are Alanine.

In some embodiments the peptide comprises FRIMAVLAS (SEQ ID NO: 2), IRIMATLAI (SEQ ID NO: 4), FRIMAVLAI (SEQ ID NO: 75), or IRIMAVLAS (SEQ ID NO: 76) or combinations thereof.

The peptide in some embodiments has 9-20 amino acids.

In other embodiments the CLIP inhibitor comprises a peptide selected based on the subject's HLA-DR allele.

In some embodiments, the method further comprises administering an immune checkpoint modulator to the subject. In one embodiment, the immune checkpoint modulator is an inhibitory checkpoint polypeptide. In another embodiment, the inhibitory checkpoint polypeptide inhibits PD1, PD-L1, CTLA4, or a combination thereof. In some embodiments, the checkpoint inhibitor polypeptide is an antibody. In other embodiments, the inhibitory checkpoint polypeptide is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, and a combination thereof. In some embodiments, the checkpoint inhibitor polypeptide is an anti-PD-L1 antibody selected from atezolizumab, avelumab, or durvalumab. In another embodiment, the checkpoint inhibitor polypeptide is an anti-CTLA-4 antibody selected from tremelimumab or ipilimumab. In other embodiments, the checkpoint inhibitor polypeptide is an anti-PD1 antibody selected from nivolumab or pembrolizumab.

In one embodiment, the immune checkpoint modulator is administered at a dosage level sufficient to deliver 100-300 mg to the subject. In some embodiments, the immune checkpoint modulator is administered at a dosage level sufficient to deliver 200 mg to the subject. In some embodiments, the immune checkpoint modulator is administered by intravenous infusion. In one embodiment, the immune checkpoint modulator is administered to the subject twice, three times, four times or more. In some embodiments, the immune checkpoint modulator is administered to the subject on the same day as the CLIP inhibitor administration.

In some embodiments the methods further involve administering to the subject an MHC binding agent, anti-cancer therapy and/or an autophagy inhibitor.

The autophagy inhibitor in some embodiments is a 4-aminoquinoline or a pharmaceutically acceptable salt or prodrug thereof. The autophagy inhibitor may be, for instance, chloroquine, 2-hydroxychloroquine, amodiaquine, mondesethylchloroquine, quinoline phosphate, or chloroquine phosphate or mixtures thereof.

A composition of a CLIP inhibitor and an immune checkpoint modulator is provided according to other aspects of the invention. Optionally, the composition further includes a carrier.

A kit is provided according to other aspects of the invention. The kit includes one or more containers housing a CLIP inhibitor and/or immune checkpoint modulator and instructions for administering to a subject the CLIP inhibitor and/or the immune checkpoint modulator.

In other aspects the invention is any of the compositions or combinations of compositions described herein for use in the treatment of a cancer or in the manufacture of a medicament for the treatment of cancer.

In some aspects the invention is a method of treating a subject having cancer, by administering to the subject an MHC class II inducing agent, and a checkpoint inhibitor in an effective amount to treat the cancer. The method may further comprise administering an MHC class II specific CLIP inhibitor to the subject. In some embodiments the MHC class II inducing agent is interferon gamma (IFNγ) or an HDAC inhibitor. In some embodiments the MHC class II inducing agent is a riminophenazine, clofazimine, B669, opsonised yeast, IL3, TNFalpha (TNFα), GM-CSF, CpG oligonucleotide, LPS, Poly I:C, Peptidoglycan, IL4 and/or IL12.

In other embodiments the MHC class II inducing agent is administered prior to the checkpoint inhibitor. In yet other embodiments the MHC class II inducing agent is administered prior to the MHC class II specific CLIP inhibitor.

The subject may be treated with the checkpoint inhibitor and the inducing agent and/or the MHC class II specific CLIP inhibitor in any combination or over any time frame. For instance the subject may be treated with the checkpoint inhibitor within 1, 2, 3, 4, 5, or 6 months of the CLIP inhibitor and the inducing agent.

In some embodiments the subject is administered at least 2, 3, 4, 5, 6, 7, 8, 9, or 10 doses of the CLIP inhibitor and the inducing agent. In other embodiments the subject is administered at least 2, 3, 4, 5, 6, 7, 8, 9, or 10 doses of the checkpoint inhibitor.

In some embodiment the CLIP inhibitor and the inducing agent are administered on a regular basis to the subject. In other embodiments the CLIP inhibitor and the inducing agent are administered to the subject daily or the CLIP inhibitor and the inducing agent are administered to the subject every other day. In some embodiments the CLIP inhibitor and the inducing agent are administered to the subject weekly.

In other aspects the invention is a method of detecting a MHC class II expressing tumor cell in a subject by obtaining a sample of tumor cells from a subject, detecting whether the tumor cells express MHC class II by performing an assay to detect MHC class II expression in the tumor cells.

In some embodiments the assay involves detecting expression of cell surface MHC class II using an antibody. In other embodiments the assay involves detecting expression of MHC class II RNA. In yet other embodiments the assay involves co-incubating the tumor cells with T cells and determining whether the T cell is activated.

The method may also further include contacting the tumor cells with an MHC class II inducing agent and measuring a level of MHC class II expression in the tumor cells after treatment with the MHC class II inducing agent.

The method may also further include administering a checkpoint inhibitor to the subject if the tumor cells express a baseline or greater level of MHC class II.

In other embodiments the method may also further include administering to the subject an isolated MHC class II specific CLIP inhibitor.

In other embodiments the method may also further include administering an MHC class II inducing agent to the subject if the tumor cells express less than a baseline level of MHC class II.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B. A set of graphs depicting the effect of CLIP inhibitors delivered in vivo on B cells (1A) versus CLIP+B cells (1B). Mice were injected with Complete Freunds adjuvant at time 0, without or with peptide injected 3 times per week starting at day 3 post CFA. Cells were harvested, stained, and counted. Flow cytometric analysis was used to determine the level of cell surface MHC and CLIP.

FIGS. 2A-2C. Flow cytometry data showing that when activated, MHC class II expressing cells express MHC class II invariant peptide (CLIP), the cells co-express PDL1. The following markers were examined: FSC-A (2A), CD3 (2B) and PD-L1 (2C). This result suggests that both CLIP and PDL-1 prevent, cell death.

FIGS. 3A-3B. A set of bar graphs demonstrating that CLIP Protects Cells from MHC Class II-mediated death. MHC Class-II-mediated Cell Death Can be Restored by Competitive Replacement of CLIP with CLIP inhibitors (3A). The absence of CLIP (CLIP knock outs) leaves the cells susceptible to MHC Class II-mediated cell death (3B).

### DETAILED DESCRIPTION

For many years melanoma has been a disease with very high mortality. The advent of check-point inhibitor (ICI) therapies for melanoma within the last few years has marked a turning point in the history of immunotherapy, increasing cancer free survival rates by roughly 30-40%. Checkpoint inhibitors unlock the “brakes” that are placed on an effective anti-tumor immune response by certain molecular interactions between the immune system and the cancer. However, in many patients those brakes can't be unlocked. It has been found, quite unexpectedly, that CLIP inhibitors can effectively modulate immune cells in order to enhance the reaction to checkpoint inhibitors. These methods could effectively bridge the gap between those that respond to ICI therapy (30% of patients) and those that don't (70% of patients) by enabling specific tumor cell recognition and by targeting MHC class II-mediated tumor cell death. The CLIP inhibitors are an immune modulating therapy that, in conjunction with the cell's expression of MHCII, causes the death of unwanted cells, including tumor cells

Recognition of CLIP inhibitors and MHCII has 2 major effects. First, T cell activation, a key component of an immune response, requires recognition of MHCII and antigen, and co-stimulation. Second, engagement of CLIP inhibitors and MHCII can “without brakes” cause death of unwanted cells in conjunction with T cell activation. Thus, custom CLIP inhibitors in the MHC binding cleft provides a novel mechanism for eliminating cancer cells.

The methods, in some aspects, involve a combination therapy. The combination involves the use of a CLIP inhibitor (such as a peptide inhibitor) to increase the percentage of patients that respond to checkpoint inhibitors by combining the activity of checkpoint inhibitors with a CLIP inhibitor capable of directly activating a program of tumor cell death. The combination therapy involves the treatment of a subject with both therapies. The treatment may occur at the same times or at different times. The compositions may be delivered in one formulation or, preferably in separate formulations.

Thus the invention, in aspects, involves new methods for treating cancer. The novel use of selective immune cell depletion using CLIP inhibitors (i.e. a death-inducing peptide) or by therapeutic use of selective immune cell depletion using highly specific therapeutic antibodies as methods for modulating the immune system to enhance checkpoint inhibitory activity is an important component of the invention. A number of small amino acid peptides that are predicted to bind in the peptide-binding groove of MHC class II alleles with a greater binding constant than the invariant MHC-associated peptide (CLIP) have been identified and synthesized. These CLIP inhibitors can target pro-inflammatory, MHCII-expressing immune cells by causing MHCII-mediated death of immune cells. MHCII-mediated cell death has been described as a part of T cell recognition resulting in both T cell activation and the death of antigen presenting cells. These peptides or depleting antibodies can be used to eliminate the expanded subsets of peripherally activated immune cells as novel combination therapies for cancer.

A CLIP inhibitor as used herein is any molecule that reduces the association of a CLIP molecule with an MHC molecule, for instance, by binding to the MHC and blocking the CLIP-MHC interaction or inhibiting the expression of CLIP. The CLIP inhibitor may function by displacing CLIP from the surface of a CLIP molecule expressing cell. A CLIP molecule expressing cell is a cell that has MHC class I or II on the surface and includes a CLIP molecule within that MHC. Such cells include, for example, epithelial cells, endothelial cells, and cells of the vascular endothelium.

The CLIP molecule, as used herein, refers to intact CD74 (also referred to as invariant chain) or intact CLIP, as well as the naturally occurring proteolytic fragments thereof. Intact CD74 or intact CLIP refer to peptides having the sequence of the native CD74 or native CLIP respectively. The CLIP molecule is one of the naturally occurring proteolytic fragments of CD74 or CLIP in some embodiments. The CLIP molecule may be, for example, at least 90% homologous to the native CD74 or CLIP molecules. In other embodiments the CLIP molecule may be at least 91%, 92%, 93%, 94%, 95%, 96%, 97%, 98%, or 99% homologous to the native CD74 or CLIP molecules An example of native CLIP molecule is MRMATPLLM (SEQ ID NO: 1), and in three-letter abbreviation as: Met Arg Met Ala Thr Pro Leu Leu Met (SEQ ID NO: 1). An example of native CD74 molecule is MHRRRSRSCR EDQKPVMDDQ RDLISNNEQL PMLGRRPGAP ESKCSRGALY TGFSILVTLL AGQATTAYF LYQQQGRLDK LTVTSQNLQL ENLRMKLPKP PKPVSKMRMA TPLLMQALPM GALPQGPMQN ATKYGNMTED HVMHLLQNAD PLKVYPPLKG SFPENLRHLK NTMETIDWKV FESWMHHWLL FEMSRHSLEQ KPTDAPPKVL TKCQEEVSHI PAVHPGSFRP KCDENGNYLP LQCYGSIGYC WCVFPNGTEV PNTRSRGHHN CSESLELEDP SSGLGVTKQD LGPVPM (SEQ ID NO: 88).

CLIP inhibitors include peptides and small molecules that can replace CLIP. In some embodiments the CLIP inhibitor is a peptide. A number of peptides useful for displacing CLIP molecules are described in U.S. Patent application Ser. No. 12/508,543 (publication number US-2010-0166782-A1); Ser. No. 12/739,459 (publication number US-2011-0118175) and Ser. No. 12/508,532 (publication number US-2010-0166789-A1) each of which is herein specifically incorporated by reference. For instance a number of these peptides are “thymus nuclear protein (TNP)” peptides.

CLIP inhibitors include for instance but are not limited to competitive CLIP fragments, MHC class II binding peptides and peptide mimetics. Thus, the CLIP inhibitor includes peptides and peptide mimetics that bind to MHC class II and displace CLIP. For instance, an isolated peptide comprising X₁RX₂X₃X₄X₅LX₆X₇ (SEQ ID NO: 3), wherein each X is an amino acid, wherein R is Arginine, L is Leucine and wherein at least one of X₂ and X₃ is Methionine, wherein the peptide is not N-MRMATPLLM-C (SEQ ID NO: 1), and wherein the peptide is a CLIP displacer is provided according to the invention. X refers to any amino acid, naturally occurring or modified. In some embodiments the Xs referred to the in formula X₁RX₂X₃X₄X₅LX₆X₇ (SEQ ID NO: 8) have the following values:

X₁ is Ala, Phe, Met, Leu, Be, Val, Pro, or Trp

X₂ is Ala, Phe, Met, Leu, Be, Val, Pro, or Trp

X₃ is Ala, Phe, Met, Leu, Be, Val, Pro, or Trp.

wherein X₄ is any

X₅ is Ala, Phe, Met, Leu, Be, Val, Pro, or Trp

X₆ is any

X₇ is Ala, Cys, Thr, Ser, Gly, Asn, Gln, Tyr.

The peptide preferably is FRIM X₄VLX₆S (SEQ ID NO: 6), such that X₄ and X₆ are any amino acid and may be Ala. Such a peptide is referred to as FRIMAVLAS (SEQ ID NO: 2), also referred to as TPP. Other preferred peptides of the invention include: IRIMATLAI (SEQ ID NO: 4), FRIMAVLAI (SEQ ID NO: 75), and IRIMAVLAS (SEQ ID NO: 76).

The minimal peptide length for binding HLA-DR is 9 amino acids. However, there can be overhanging amino acids on either side of the open binding groove. For some well-studied peptides, it is known that additional overhanging amino acids on both the N and C termini can augment binding. Thus the peptide may be 9 amino acids in length or it may be longer. For instance, the peptide may have additional amino acids at the N and/or C terminus. The amino acids at either terminus may be anywhere between 1 and 100 amino acids. In some embodiments the peptide includes 1-50, 1-20, 1-15, 1-10, 1-5 or any integer range there between. When the peptide is referred to as “N-FRIMAVLAS-C” (SEQ ID NO: 2) or “N-X₁RX₂X₃X₄X₅LX₆X₇-C” (SEQ ID NO: 8) the —C and —N refer to the terminus of the peptide and thus the peptide is only 9 amino acids in length. However the 9 amino acid peptide may be linked to other non-peptide moieties at either the —C or —N terminus or internally.

Other peptides useful as CLIP inhibitors, including some TNP peptides and synthetic peptides are shown in Table 1.

TABLE 1

| Amino Acid | SEQ |
|---|---|
| Sequence | ID NO: |
| LVQNDTLLQ | 10 |
| VVSTQTALA | 11 |
| IMNSFVNDI | 12 |
| MGIMKSFVN | 13 |
| MGIMNSFVN | 14 |
| VLIAFSQYL | 15 |
| IMNSFVNDL | 16 |
| IMKSFVNDI | 17 |
| IQGITKPAI | 18 |
| VTAMDVVYA | 19 |
| YGFQNALIV | 20 |
| LVNELTEFA | 21 |
| FQNALIVRY | 22 |
| MSIMNSFVN | 23 |
| LVLIAFSQY | 24 |
| VQNDTLLQV | 25 |
| MGNMNSFVN | 26 |
| FQSAIKLVD | 27 |
| VAFVDKCCA | 28 |
| LVVSTQTAL | 29 |
| VFLENVIRD | 30 |
| LIAFSQYLQ | 31 |
| FQSAAIGAL | 32 |
| MDIMNSFVN | 33 |
| IKLVDFQDA | 34 |
| VMENFVAFV | 35 |
| YLQQCPFDE | 36 |
| VLPNIQAVL | 37 |
| VEPSDTIEN | 38 |
| FFQSAIKLV | 39 |
| IQAVLLPKK | 40 |
| IAFSQYLQQ | 41 |
| FLGSFLYEY | 42 |
| FVNDIFERI | 43 |
| LPNIQAVLL | 44 |
| LLPGELAKH | 45 |
| FVAFVDKCC | 46 |
| LKPDPNTLC | 47 |
| MENFVAFVD | 48 |
| LFGDELCKV | 49 |
| VTIAQGGVL | 50 |
| MKSFVNDIF | 51 |
| LFTFHADIC | 52 |
| FVNDLFERL | 53 |
| IAQGGVLPN | 54 |
| LGSFLYEYS | 55 |
| FVDKCCAAD | 56 |
| LFEDTNLCA | 57 |
| VNFAEFSKK | 58 |
| MNSFVNDIF | 59 |
| MNSFVNDLF | 60 |
| LVDEPQNLI | 61 |
| MDVVYALKR | 62 |
| LLLPGELAK | 63 |
| LTPDETYVP | 64 |
| LQNEIDVSS | 65 |
| LVDFQDAKA | 66 |
| VGLFEDTNL | 67 |
| LGLIYEETR | 68 |
| ILGLIYEET | 69 |
| IDVSSREKS | 70 |
| LHTLFGDEL | 71 |
| LVGLFEDTN | 72 |
| IAQDFKTDL | 73 |
| FHADICTLP | 74 |

In some instances the peptides may be mixed with cystatin A and/or histones and in other instances the composition is free of cystatin A or histones. Histone encompasses all histone proteins including HI, H2A, H2B, H3, H4 and H5.

The peptide may be cyclic or non-cyclic. Cyclic peptides in some instances have improved stability properties. Those of skill in the art know how to produce cyclic peptides.

The peptides may also be linked to other molecules. The peptide and molecule may be linked directly to one another (e.g., via a peptide bond); linked via a linker molecule, which may or may not be a peptide; or linked indirectly to one another by linkage to a common carrier molecule, for instance.

Thus, linker molecules (“linkers”) may optionally be used to link the peptide to another molecule. Linkers may be peptides, which consist of one to multiple amino acids, or non-peptide molecules. Examples of peptide linker molecules useful in the invention include glycine-rich peptide linkers (see, e.g., U.S. Pat. No. 5,908,626), wherein more than half of the amino acid residues are glycine. Preferably, such glycine-rich peptide linkers consist of about 20 or fewer amino acids.

The peptide for instance, may be linked to a PEG or TEG molecule. Such a molecule is referred to as a PEGylated or TEGylated peptide.

In certain embodiments, the CLIP inhibitor is an inhibitory nucleic acid such as a small interfering nucleic acid molecule such as antisense, RNAi, or siRNA oligonucleotide to reduce the level of mature CLIP molecule (CD74) expression. The nucleotide sequences of CD74 molecules are well known in the art and can be used by one of skill in the art using art recognized techniques in combination with the guidance set forth herein to produce the appropriate siRNA molecules. An example of a CD74 nucleic acid molecule is shown in SEQ ID NO: 77.

Small interfering nucleic acid (siNA) include, for example: microRNA (miRNA), small interfering RNA (siRNA), double-stranded RNA (dsRNA), and short hairpin RNA (shRNA) molecules. An siNA useful in the invention can be unmodified or chemically-modified. An siNA of the instant invention can be chemically synthesized, expressed from a vector or enzymatically synthesized. Such methods are well known in the art. Exemplary single stranded regions of siRNA for CLIP are shown below. The invention contemplates others as well.

(SEQ ID NO: 78)
GGUAGUAAUUAGAACAAAA
(SEQ ID NO: 79)
GGUUCACAUUAGAAUAAAA
(SEQ ID NO: 80)
GAACAAAAAAAAAAAAAAA
(SEQ ID NO: 81)
CAAAAAAAAAAAAAAAAAA
(SEQ ID NO: 82)
AGAACAAAAAAAAAAAAAA
(SEQ ID NO: 83)
ACAAAAAAAAAAAAAAAAA
(SEQ ID NO: 84)
GUAAUUAGAACAAAAAAAA
(SEQ ID NO: 85)
CAUGGUUCACAUUAGAAUA
(SEQ ID NO: 86)
GUAGUAAUUAGAACAAAAA
(SEQ ID NO: 87)
GGCUUUUCUAGCCUAUUUA

In one embodiment, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

Other inhibitor molecules that can be used include ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9,1996). Multitarget ribozymes (connected or shotgun) have been suggested as a means of improving efficiency of ribozymes for gene suppression (Ohkawa et al., Nucleic Acids Symp Ser. (29):121-2, 1993).

In some aspects of the invention the tumor cell does not express MHC class II or expresses low levels of MHC class II. In order to accomplish the methods of the invention the tumor or subject may be treated with an MHC class II inducing agent in order to promote the expression of MHC class II on the tumor cell. The level of MHC class II on a tumor may be assessed by an assay in order to determine a baseline or threshold level of MHC class II expression on the tumor cell. A baseline or threshold level is a minimal amount of MHC that can induce MHC class II mediated death. The amount can be determined in a particular tumor cell using methods known in the art.

In some embodiments the subject may be treated with an MHC class II inducing agent and a checkpoint inhibitor and optionally a CLIP inhibitor regardless of the MHC class II status of the tumor cell. It is not required that the expression level of MHC class II on the tumor cell be determined prior to treatment.

An MHC class II inducing agent is a compound that induces the expression of MHC class II on a tumor cell that in the absence of the treatment did not express or expressed MHC class II only below threshold levels. In some embodiments the MHC class II inducing agent is interferon-gamma (IFN-γ), a retinoic acid receptor-alpha/beta-selective retinoid such as Am80 (tamibarotene), a Histone deacetylase (HDAC) inhibitor, the riminophenazines, clofazimine, B669, IL3, TNFα, GM-CSF, CpG oligonucleotide, LPS, Poly I:C, Peptidoglycan, IL4, IL12 or an IFN-γ inducing agent such as an immunostimulatory nucleic acid (i.e. a C-class CpG oligonucleotide).

HDAC inhibitors (HDACi), such as trichostatin A (TSA) and valproic acid, which have broad HDAC specificity have been demonstrated to induce MHC class II, CD40, MICA, and MICB genes by epigenetic modulation. This induction of MHC and costimulatory molecules on tumors has been shown to elicit effective antigen presentation.

Different tumor cells respond differently to different factors for inducing MHC expression. The skilled artisan may select an appropriate inducing agent based on the knowledge in the art or routine experimentation which tests the ability of a known inducing agent to promote MHC class II expression on a particular tumor cell. For instance, it is known that cells such as HeLa produce high levels of MHC class II in response to IFN-γ but low levels following treatment with HDACi. Some tumor cells, do not respond to IFN-γ but express MHC class II after HDACi treatment. Other tumor cells, such as colon, have been shown to respond to IFN-γ activation of MHC class II requiring CIITA and also TSA-activated class II in the absence of CIITA.

The invention involves methods for treating a subject. A subject shall mean a human or vertebrate mammal including but not limited to a dog, cat, horse, goat and primate, e.g., monkey. Thus, the invention can also be used to treat diseases or conditions in non-human subjects. Preferably the subject is a human. In some embodiments the subject has a cancer.

In some embodiments, a subject may be diagnosed with, or otherwise known to have, a disease or bodily condition associated with cancer, as described herein. Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

In some preferred embodiments of the invention the CLIP inhibitors are administered with a T cell activator such as an immune checkpoint modulator. Immune checkpoint modulators include both stimulatory checkpoint molecules and inhibitory checkpoint molecules i.e., an anti-CTLA4 and anti-PD1 antibody.

A checkpoint inhibitor is a compound that inhibits a protein in the checkpoint signalling pathway. Proteins in the checkpoint signalling pathway include for example, PD-1, PD-L1, PD-L2, LAG3, TIM3, and CTLA-4. Checkpoint inhibitors are known in the art. For example, the checkpoint inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. The checkpoint inhibitor may be an antibody or antigen binding fragment thereof.

Stimulatory checkpoint inhibitors function by promoting the checkpoint process. Several stimulatory checkpoint molecules are members of the tumor necrosis factor (TNF) receptor superfamily—CD27, CD40, OX40, GITR and CD137, while others belong to the B7-CD28 superfamily—CD28 and ICOS. OX40 (CD134), is involved in the expansion of effector and memory T cells. Anti-OX40 monoclonal antibodies have been shown to be effective in treating advanced cancer. MEDI0562 is a humanized OX40 agonist. GITR, Glucocorticoid-Induced TNFR family Related gene, is involved in T cell expansion Several antibodies to GITR have been shown to promote an anti-tumor responses. ICOS, Inducible T-cell costimulator, is important in T cell effector function. CD27 supports antigen-specific expansion of naïve T cells and is involved in the generation of T and B cell memory. Several agonistic anti-CD27 antibodies are in development. CD122 is the Interleukin-2 receptor beta sub-unit. NKTR-214 is a CD122-biased immune-stimulatory cytokine.

Inhibitory checkpoint molecules include but are not limited to PD-1, TIM-3, VISTA, A2AR, B7-H3, B7-H4, BTLA, CTLA-4, IDO, KIR and LAG3. CTLA-4, PD-1 and its ligands are members of the CD28-B7 family of co-signaling molecules that play important roles throughout all stages of T-cell function and other cell functions. CTLA-4, Cytotoxic T-Lymphocyte-Associated protein 4 (CD152) is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response. Pembrolizumab (formerly MK-3475 and lambrolizumab, trade name Keytruda) is a human antibody used in cancer immunotherapy. It targets the PD-1 receptor.

IDO, Indoleamine 2,3-dioxygenase, is a tryptophan catabolic enzyme, which suppresses T and NK cells, generates and activates Tregs and myeloid-derived suppressor cells, and promotes tumor angiogenesis. TIM-3, T-cell Immunoglobulin domain and Mucin domain 3, acts as a negative regulator of Thl/Tcl function by triggering cell death upon interaction with its ligand, galectin-9. VISTA, V-domain Ig suppressor of T cell activation.

The checkpoint inhibitor is a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands. In some embodiments the anti-PD-1 antibody is BMS-936558 (nivolumab). In other embodiments the anti-CTLA-4 antibody is ipilimumab (trade name Yervoy, formerly known as MDX-010 and MDX-101).

In some embodiments the checkpoint inhibitor is a targeted therapy. The targeted therapy may be a BRAF inhibitor such as vemurafenib (PLX4032) or dabrafenib. The BRAF inhibitor may be PLX 4032, PLX 4720, PLX 4734, GDC-0879, PLX 4032, PLX-4720, PLX 4734 and Sorafenib Tosylate. BRAF is a human gene that makes a protein called B-Raf, also referred to as proto-oncogene B-Raf and v-Raf murine sarcoma viral oncogene homolog B1. The B-Raf protein is involved in sending signals inside cells, which are involved in directing cell growth. Vemurafenib, a BRAF inhibitor, was approved by FDA for treatment of late-stage melanoma.

The checkpoint inhibitor in other embodiments is an OX40L. OX40 is a member of the tumor necrosis factor/nerve growth factor receptor (TNFR/NGFR) family. OX40 may play a role in T-cell activation as well as regulation of differentiation, proliferation or apoptosis of normal and malignant lymphoid cells.

As used herein, the term treat, treated, or treating when used with respect to a disorder refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy.

When used in combination with the therapies of the invention the dosages of known therapies may be reduced in some instances, to avoid side effects.

The CLIP inhibitor can be administered in combination with the checkpoint inhibitors (or other T cell activators) and such administration may be simultaneous or sequential. When the checkpoint inhibitors are administered simultaneously they can be administered in the same or separate formulations, but are administered at the same time. The administration of the checkpoint inhibitors and the CLIP inhibitor can also be temporally separated, meaning that the checkpoint inhibitors are administered at a different time, either before or after, the administration of the CLIP inhibitor. The separation in time between the administration of these compounds may be a matter of minutes or it may be longer.

The active agents of the invention are administered to the subject in an effective amount for treating disorders such as cancer. An “effective amount”, for instance, is an amount necessary or sufficient to realize a desired biologic effect. An effective amount for treating cancer may be an amount sufficient to reduce proliferation rates or growth of a tumor. According to some aspects of the invention, an effective amount is that amount of a compound of the invention alone or in combination with another medicament, which when combined or co-administered or administered alone, results in a therapeutic response to the disease, either in the prevention or the treatment of the disease. The biological effect may be the amelioration and or absolute elimination of symptoms resulting from the disease. In another embodiment, the biological effect is the complete abrogation of the disease, as evidenced for example, by the absence of a symptom of the disease.

The effective amount of a compound of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma or lymph fluid (derived from lymphatic tissues, lymph nodes, or the interstitium), concentration range that includes the IC₅₀ (i.e., the concentration of the test compound that achieves a half-maximal inhibition of symptoms) as determined in cell culture. Such information can be used to more accurately determine useful doses in humans. Levels in plasma or in lymph fluids may be measured, for example, by high performance liquid chromatography.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

Multiple doses of the molecules of the invention are also contemplated. In some instances, when the molecules of the invention are administered with another therapeutic, a sub-therapeutic dosage of either or both of the molecules may be used. A “sub-therapeutic dose” as used herein refers to a dosage which is less than that dosage which would produce a therapeutic result in the subject if administered in the absence of the other agent.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference).

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods of the invention, the compound may be administered in a pharmaceutical composition. In general, a pharmaceutical composition comprises the compound of the invention and a pharmaceutically-acceptable carrier. Pharmaceutically-acceptable carriers for peptides, monoclonal antibodies, and antibody fragments are well-known to those of ordinary skill in the art. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, the preferred vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Therapeutic formulations of the compounds, i.e., peptides, small molecules, nucleic acids or antibodies may be prepared for storage by mixing a compounds having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The compounds may be administered directly to a cell or a subject, such as a human subject alone or with a suitable carrier. Additionally, a peptide may be delivered to a cell in vitro or in vivo by delivering a nucleic acid that expresses the peptide to a cell. Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, lipo some-mediated transfection, and the like. For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half-life, and the like. Polymeric delivery systems also have been used successfully to deliver nucleic acid molecules into cells, as is known by those skilled in the art. Such systems even permit oral delivery of nucleic acid molecules.

The peptide of the invention may also be expressed directly in mammalian cells using a mammalian expression vector. Such a vector can be delivered to the cell or subject and the peptide expressed within the cell or subject. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the α-fetoprotein promoter.

As used herein, a “vector” may be any of a number of nucleic acid molecules into which a desired sequence may be inserted by restriction and ligation for expression in a host cell. Vectors are typically composed of DNA although RNA vectors are also available. Vectors include, but are not limited to, plasmids, phagemids and virus genomes. An expression vector is one into which a desired DNA sequence may be inserted by restriction and ligation such that it is operably joined to regulatory sequences and may be expressed as an RNA transcript.

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of cancer.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise proces sable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1: CLIP and PDL-1 Prevent Cell Death and that Suppression is Overcome Using CLIP Inhibitors

CD4+ T cell recognition of antigenic peptides associated with Major Histocompatibility Complex-encoded class II molecules (MHCII) is required for CD4+ T cell activation, along with co-stimulatory interactions that either stimulate or inhibit the resulting T cell response. T cell activation can, but does not always, result in cell death of the successful antigen presenting cell (APC). Our recent studies indicate that when cells are activated by Toll-like receptor (TLR) or by danger signals, the antigen binding groove of MHC class II in some APC, including B cells, is filled with CLIP that prevents cell death of the MHC class II expressing APC.

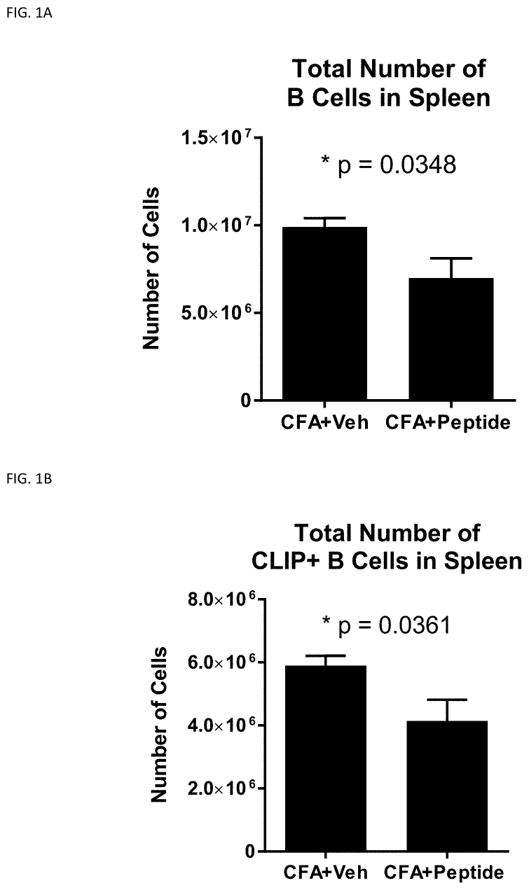

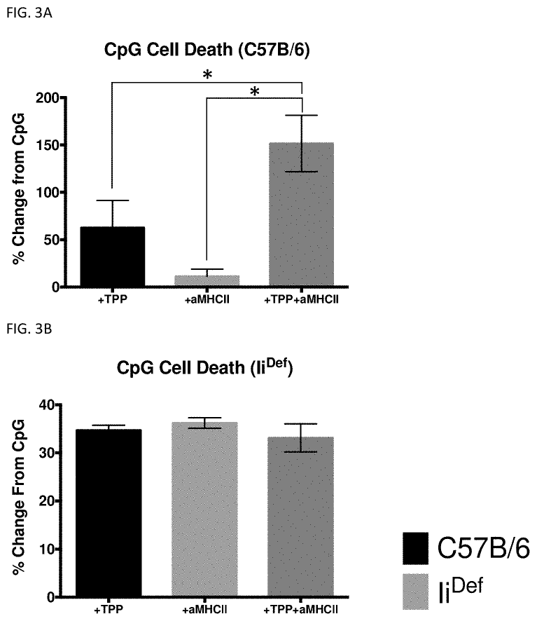

The effect of CLIP inhibitors was examined in MHC class II expressing B cells in order to demonstrate the role of CLIP in preventing cell death (FIGS. 1 and 3). In a first experiment, animals were injected with complete Freunds adjuvant (CFA) that contains mycobacterial DNA (a well-established TLR activator), or with CFA followed by the competitive antagonist peptide (CLIP inhibitor, SEQ ID NO. 2). Spleens were harvested, stained with CD19 for the detection of B cells, versus CLIP as an indicator of CD19+ cells expressing CLIP. The data is shown in FIGS. 1A-1B. The effect of CLIP inhibitors delivered in vivo on B cells (1A) versus CLIP+B cells (1B) is presented in the bar graphs.

To test the hypothesis that CLIP in the groove prevents MHCII-mediated cell death, we utilized animals that are deficient in CD74 expression (Ii Def mice), thus lacking both the precursor CD74 molecule as well as its proteolytic product, CLIP. In this experiment, treatment with TLR9 agonist CpG in C57/B6 mice results in cell surface CLIP expression and resistance to MHCII-mediated cell death, unless the CpG activated cells are treated with peptide (labelled as TPP for targeted predicted peptide) followed by treatment with antibodies to MHCII. In contrast, when cells from the CD74 deficient animal are refractile to any increase in cell death following CpG treatment, FIGS. 3A and 3B. The bar graphs demonstrate that MHC Class-II-mediated cell death can be restored by competitive replacement of CLIP with CLIP inhibitors (3A). The absence of CLIP (CLIP knock outs) leaves the cells susceptible to MHC Class II-mediated cell death (3B).

The nature of the CD4+ T cell response, i.e. the cytokines produced and the effector function of the CD4+ T cell, is shaped in large part by the second requirement for the T cell's activation, co-stimulation. Costimulation can be stimulatory or, in the case of certain “immune check point” receptor:ligand pairs, inhibitory. The fate of the APC can also be determined by the balance between T cell activation and/or inhibition and the consequence can be either survival of the APC or T cell activation can cause MHCII mediated cell death of the APC.

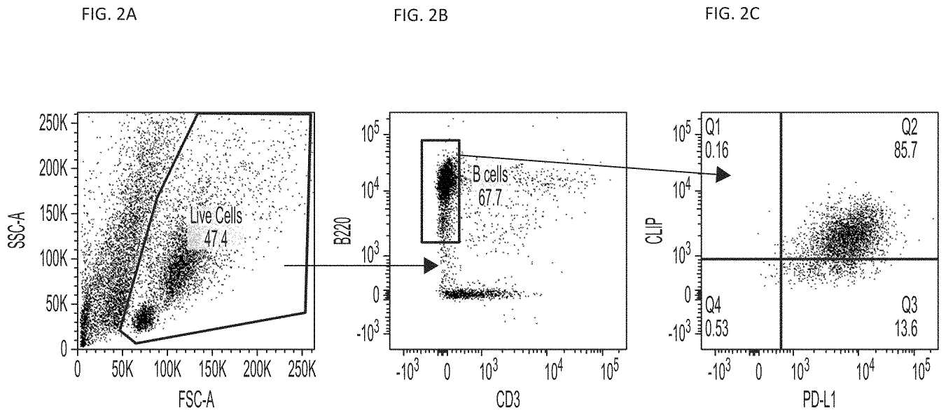

Furthermore, we have found that co-incident with cell surface CLIP expression on activated and proliferating cells, is the cell surface expression of PD-1, a well characterized inhibitor of conventional T cell activation. Thus, it appears that protection from cell death may be conferred by a number of key cell surface changes that prevent the activated or proliferating cell from being eliminated, FIG. 2, helping explain the resistance of some cancer cells to treatment.

MHC class II expressing cells were examined for expression of PDL1, CD3 and FSC-A using flow cytometry. The data is presented in FIGS. 2A-2C. The data show that when activated, MHC class II expressing cells express MHC class II invariant peptide (CLIP) and the cells co-express PDL1. The data is represented by staining of FSC-A (2A), CD3 (2B) and PD-L1 (2C).

### Example 2: Assays and Therapeutic Interventions

Because of undesirable and occasionally serious side effects of some checkpoint inhibitors the susceptibility of the patient to the drug may be determined, according to the methods of the invention, before treatment. An in-vitro assay on biopsied tumor cells can determine if checkpoint inhibitor drugs would be effective in treating the tumor alone. Other versions of the assay can show whether the checkpoint inhibitor drugs in combination with other therapies would be more effective. This allows an individual therapeutic treatment plan to be developed.

Initially an in vitro assay system to determine the susceptibility of an individual's unwanted and/or tumor cells to MHCII-mediated cell death may be performed. Once susceptibility is determined an individualized treatment plan involving promoting MHC class II death and checkpoint inhibitor therapy is used. Ideally the treatment will involve administration of a CLIP inhibitor such as a peptide (i.e. SEQ ID NO 2) therapy as a supplement to “immune check point inhibitor” (ICI) therapies in those individuals that are non-responsive to current ICI therapies. One assay involves cell culture and flow cytometry, and flow cytometric analysis, that can assess the efficacy of the peptide therapy. The rationale for this approach is based on identifying an individual's distinct profile of immune response genes and the MHCII expression profiles on unwanted, i.e. tumor cells.

Establish a Standardized Cell Culture System of Well-Established Tumor Cell Lines in which to Test Susceptibility to Peptide-Induced, MHCII-Mediated Cell Death.

To confirm the expression of MHCII staining with fluorochrome-conjugated anti-MHCII antibodies, followed by flow cytometric analysis may be performed. The cells are stained for a panel of cell surface molecules, including CD19, CD80 (B7.1), CD86 (B7.2), CD74, MHCII associated invariant peptide (CLIP), and the family of immune check points, including PD1, B7-H2, and B7-H4. As a starting concentration, the cultured cells are treated with a competitive antagonist peptide (CAP) at a concentration of 5 μg/ml, as established from preliminary studies as the optimum concentration for competitive displacement of peptides in the peptide binding groove of MHCII in B cells. These studies may be followed thereafter by a dose response of peptide concentrations ranging from 0.5 μg/ml to 50 μg/ml. The cells are harvested and stained to determine if peptide treatment reduces the amount of CLIP per cell using flow cytometric analysis. To characterize these parameters, including levels of MHCII and other cell surface characteristics, the cells are treated with the competitive antagonist peptide at the previously determined optimum concentration, presumably 5 μg/ml, followed by treatment with anti-MHCII antibody as a surrogate for TCR engagement. Susceptibility to MHCII-mediated cell death is assessed by treating the cells with peptide, followed by treatment with anti-MHCII antibody for periods ranging between 3 and 24 hours to determine the optimum kinetics for inducing cell death. The cells are then harvested and counted, using either a Cell-O-meter™ or hemocytometer counts (using a viability dye, such as Trypan blue), as a measure of recovered cell numbers, and using flow cytometry to obtain a percent of live versus dead cells following treatment. A variety of MCH class II inducing agents, such as IFNγ or Toll-like receptor (TLR) agonists, may be used to increase or to induce cell surface expression of MHCII on the tumor cell surfaces. The capability of CAP peptide treatment to promote MHCII-mediated cell death may be assessed.

Determine if Cell Surface Expression of CD74 or its Proteolytic Product(s) CLIP can be Used as a Biomarker to Predict Susceptibility to MHCII-Mediated Cell Death.

Because expression of CD74 and/or its proteolytic cleavage product CLIP are associated with cell survival and with inflammation, expression of CD74 or CLIP on the tumor cell surface serves as a biomarker(s) predicting resistance to MHCII-mediated cell death may be assessed. The cell surface expression of CD74, as a known tumor survivor factor, and its cleavage product CLIP as potential indicators of resistance to MHCII-mediated cell death are assessed. The data demonstrate that tumors that express MHCII, coexpress both cell surface CLIP as well as the survival molecule PD1 and that CLIP in the peptide binding groove prevents MHCII-mediated cell death. Taken together these data suggest that cell surface CD74 as well as cell surface CLIP may be indicators of a survival strategy that blocks MHCII mediated cell death and may provide a biomarker that predicts the need for a further active such as the CAP peptide treatment to enable MHCII-mediated cell death.

Establish an Assay System to Test Freshly Excised Human Tumor Tissue for Susceptibility to MHCII Mediated Cell Death.

Isolates of human tumors may be obtained from a human tumor bank, and stained for the expression of MHCII. If MHC class II is not expressed or negligibly expressed, the cells may be treated with MHCII inducing agents, and determines if CAP treatment enables MHCII-mediated cell death of that individual's tumor cells. The stored tumor tissues will have been resuspended as single cell suspensions and frozen in DMSO-containing freezing medium. Once thawed and washed, and centrifuged, single cell suspensions may be stained for MHCII, CD19, CD80 (B7.1), CD86 (B7.2), CD74, MHCII associated invariant peptide (CLIP), and check-point inhibitor family members, including PD1, B7-H2, and B7-H4. In those samples that express MHCII, the dispersed tumor cells may be cultured, treated with CAP peptide, and assessed for the capability of CAP treatment to promote MHCII-mediated cell death via both anti-MHCII treatment, as described above.

The assay may consist of an initial flow cytometric determination of expression of levels of MHC class II, CD74, CLIP, PD1, B71 and B72 levels. Further, treatment of the cells with gamma interferon to increase, or to induce, the expression of MHCII and/or CD74, followed by dose responses and kinetics of peptide treatment, and analysis of susceptibility to MHCII-mediated tumor cell death, by using antibodies to MHCII as surrogates for TCR recognition may be performed. The assessments may be made flow cytometrically and analyzed using FlowJo software.

The cell surface characteristics of each tumor line or explanted tumor cells may first be compared to assess for differences using one way Analysis of Variance (ANOVA) for continuous and normally distributed variables, Kruskal Wallis ANOVA for non-normal continuous variables, and Pearson chi-square tests for nominal/categorical variables. Post-hoc analyses will be conducted in the event of significant omnibus tests, with the appropriate analysis (Welch t-tests for continuous and normally distributed variables, Mann-Whitney U tests for non-normal continuous variables, and Pearson chi-square tests for nominal/categorical variables) to elucidate differences. These variables may be evaluated as potential covariates in subsequent analytic models. Regression models will be used to test the primary aims, either through multinomial logistic regressions or generalized linear regressions. The latter will be used for the majority of the analyses given the flexibility in working with multiple distribution types. In the event that heteroscedasticity is present in these regression models, Huber-White robust standard errors will be implemented to provide unbiased standard errors and associated p-values. Two-sided p-values less than 0.05 will be considered significant; however, given that multiple hypotheses will be tested, a False Discovery Rate (FDR) will be applied. The FDR was chosen to control for false positives given that it is more powerful than traditional methods in controlling for Type I errors.

Thus, in some exemplary embodiments, a patient with melanoma is given a biopsy to collect tumor cells. Those cells are grown up in culture and different subsets of them are tested in parallel to determine their susceptibility to different treatments. One subset is stained with fluorochrome—conjugated antibodies to the immune check points such as PL1, B7-H2, and B7-H4 to determine if the cells could be susceptible to the ICI drugs. Another subset is stained with fluorochrome—conjugated antibodies to the cell surface molecules CD19, CD80 (B7.1), CD86 (B7.2), CD74, MHCII associated invariant peptide (CLIP) to determine the likelihood of MHCII mediated cell death. A further subset is treated with IFNγ or Toll-like receptor (TLR) agonists, to increase or induce the cell surface expression of MHCII and then tested with antibodies as in the first two subsets. These cell groups are further sub-divided and half are treated with CAP peptide. After a further sub-division half are treated with the proposed ICI drug. All of these cell groups are then evaluated by flow cytometry to determine what they were expressing on the surface, whether they were susceptible to ICI drug alone, whether they could be induced to be susceptible to ICI drug by administration of an MHCII inducing agent, and whether this was aided by the presence of a peptide that could displace CLIP from the binding groove.

The melanoma cells may show no evidence of MHCII expression. They may demonstrate a resistance to ICI drugs in the absence of IFNγ to promote the expression of MHCII. They may show significant mortality when exposed to ICI drugs after inducement of MHCII by IFNγ. They may show increased mortality after a combination of IFNγ, CAP and ICI drugs. This suggests a preferred therapeutic intervention of IFNγ followed by ICI drug, possibly enhanced with CAP.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method of treating a subject having cancer, comprising
administering to the subject an isolated MHC class II specific CLIP inhibitor and a checkpoint inhibitor.

2. The method of claim 1, wherein CLIP inhibitor is synthetic.

3. The method of claim 1, wherein CLIP inhibitor is a TNP peptide.

4. The method of claim 1, wherein the CLIP inhibitor is an siRNA.

5. The method of claim 1, wherein the subject has a melanoma.

6. The method of claim 1, wherein the CLIP inhibitor is administered to the subject systemically.

7. The method of claim 1, wherein the subject is treated with the checkpoint inhibitor within 8 hours of the CLIP inhibitor.

8. The method of claim 1, wherein the subject is treated with the checkpoint inhibitor within 24 hours of the CLIP inhibitor.

9. The method of claim 1, wherein the subject is treated with the checkpoint inhibitor within 1 week of the CLIP inhibitor.

10. The method of claim 1, wherein the subject is treated with the checkpoint inhibitor within 1 month of the CLIP inhibitor.

11-38. (canceled)

39. The method of claim 37, wherein the MHC class II inducing agent is interferon gamma.

40. The method of claim 37, wherein the MHC class II inducing agent is an HDAC inhibitor.

41. The method of claim 37, wherein the MHC class II inducing agent is a riminophenazine, clofazimine, B669, opsonised yeast, IL3, TNFalpha (TNFα), GM-CSF, CpG oligonucleotide, LPS, Poly I:C, Peptidoglycan, IL4 and/or IL12.

42. The method of claim 37, wherein the MHC class II inducing agent is administered prior to the checkpoint inhibitor.

43-53. (canceled)

54. A method of detecting a MHC class II expressing tumor cell in a subject comprising,
obtaining a sample of tumor cells from a subject, detecting whether the tumor cells express MHC class II by performing an assay to detect MHC class II or CD74 expression in the tumor cells or detecting the presence of CLIP in MHC.

55. The method of claim 54, wherein the assay involves detecting expression of cell surface MHC class II using an antibody.

56. The method of claim 54, wherein the assay involves detecting expression of MHC class II RNA.

57. The method of claim 54, wherein the assay involves co-incubating the tumor cells with T cells and determining whether the T cell is activated.

58. The method of claim 54, further comprising contacting the tumor cells with an MHC class II inducing agent and measuring a level of MHC class II expression in the tumor cells after treatment with the MHC class II inducing agent.

59-61. (canceled)


SEQUENCE LISTING (88 sequences)

SEQ ID NO: 1 - protein, 9 aa, Homo sapiens
MRMATPLLM

SEQ ID NO: 2 - protein, 9 aa, Artificial Sequence
  Synthetic Polypeptide
FRIMAVLAS

SEQ ID NO: 3 - protein, 9 aa, Artificial Sequence
  Synthetic Polypeptide
  misc_feature (1)..(1): Xaa can be any naturally occurring amino acid
  misc_feature (3)..(6): Xaa can be any naturally occurring amino acid
  misc_feature (3)..(4): Xaa is Met or any naturally occurring amino acid
  misc_feature (8)..(9): Xaa can be any naturally occurring amino acid
XRXXXXLXX

SEQ ID NO: 4 - protein, 9 aa, Artificial Sequence
  Synthetic Polypeptide
IRIMATLAI

SEQ ID NO: 5 - skipped

SEQ ID NO: 6 - protein, 9 aa, Artificial Sequence
  Synthetic Polypeptide
  misc_feature (5)..(5): Xaa can be any naturally occurring amino acid
  misc_feature (8)..(8): Xaa can be any naturally occurring amino acid
FRIMXVLXS

SEQ ID NO: 7 - skipped

SEQ ID NO: 8 - protein, 9 aa, Artificial Sequence
  Synthetic Polypeptide
  MISC_FEATURE (1)..(1): Xaa is Ala, Phe, Met, Leu, Ile, Val, Pro, or Trp
  MISC_FEATURE (3)..(3): Xaa is Ala, Phe, Met, Leu, Ile, Val, Pro, or Trp
  MISC_FEATURE (4)..(4): Xaa is Ala, Phe, Met, Leu, Ile, Val, Pro, or Trp
  MISC_FEATURE (5)..(5): Xaa can be any naturally occurring amino acid
  MISC_FEATURE (6)..(6): Xaa is Ala, Phe, Met, Leu, Ile, Val, Pro, or Trp
  MISC_FEATURE (8)..(8): Xaa can be any naturally occurring amino acid
  MISC_FEATURE (9)..(9): Xaa is Ala, Cys, Thr, Ser, Gly, Asn, Gln, Tyr
XRXXXXLXX

SEQ ID NO: 9 - skipped

SEQ ID NO: 10 - protein, 9 aa, Artificial Sequence
  Synthetic Polypeptide
LVQNDTLLQ

SEQ ID NO: 11 - protein, 9 aa, Artificial Sequence
  Synthetic Polypeptide
VVSTQTALA

SEQ ID NO: 12 - protein, 9 aa, Artificial Sequence
  Synthetic Polypeptide
IMNSFVNDI

SEQ ID NO: 13 - protein, 9 aa, Artificial Sequence
  Synthetic Polypeptide
MGIMKSFVN

SEQ ID NO: 14 - protein, 9 aa, Artificial Sequence
  Synthetic Polypeptide
MGIMNSFVN

SEQ ID NO: 15 - protein, 9 aa, Artificial Sequence
  Synthetic Polypeptide
VLIAFSQYL

SEQ ID NO: 16 - protein, 9 aa, Artificial Sequence
  Synthetic Polypeptide
IMNSFVNDL

SEQ ID NO: 17 - protein, 9 aa, Artificial Sequence
  Synthetic Polypeptide
IMKSFVNDI

SEQ ID NO: 18 - protein, 9 aa, Artificial Sequence
  Synthetic Polypeptide
IQGITKPAI

SEQ ID NO: 19 - protein, 9 aa, Artificial Sequence
  Synthetic Polypeptide
VTAMDVVYA

SEQ ID NO: 20 - protein, 9 aa, Artificial Sequence
  Synthetic Polypeptide
YGFQNALIV

SEQ ID NO: 21 - protein, 9 aa, Artificial Sequence
  Synthetic Polypeptide
LVNELTEFA

SEQ ID NO: 22 - protein, 9 aa, Artificial Sequence
  Synthetic Polypeptide
FQNALIVRY

SEQ ID NO: 23 - protein, 9 aa, Artificial Sequence
  Synthetic Polypeptide
MSIMNSFVN

SEQ ID NO: 24 - protein, 9 aa, Artificial Sequence
  Synthetic Polypeptide
LVLIAFSQY

SEQ ID NO: 25 - protein, 9 aa, Artificial Sequence
  Synthetic Polypeptide
VQNDTLLQV

SEQ ID NO: 26 - protein, 9 aa, Artificial Sequence
  Synthetic Polypeptide
MGNMNSFVN

SEQ ID NO: 27 - protein, 9 aa, Artificial Sequence
  Synthetic Polypeptide
FQSAIKLVD

SEQ ID NO: 28 - protein, 9 aa, Artificial Sequence
  Synthetic Polypeptide
VAFVDKCCA

SEQ ID NO: 29 - protein, 9 aa, Artificial Sequence
  Synthetic Polypeptide
LVVSTQTAL

SEQ ID NO: 30 - protein, 9 aa, Artificial Sequence
  Synthetic Polypeptide
VFLENVIRD

SEQ ID NO: 31 - protein, 9 aa, Artificial Sequence
  Synthetic Polypeptide
LIAFSQYLQ

SEQ ID NO: 32 - protein, 9 aa, Artificial Sequence
  Synthetic Polypeptide
FQSAAIGAL

SEQ ID NO: 33 - protein, 9 aa, Artificial Sequence
  Synthetic Polypeptide
MDIMNSFVN

SEQ ID NO: 34 - protein, 9 aa, Artificial Sequence
  Synthetic Polypeptide
IKLVDFQDA

SEQ ID NO: 35 - protein, 9 aa, Artificial Sequence
  Synthetic Polypeptide
VMENFVAFV

SEQ ID NO: 36 - protein, 9 aa, Artificial Sequence
  Synthetic Polypeptide
YLQQCPFDE

SEQ ID NO: 37 - protein, 9 aa, Artificial Sequence
  Synthetic Polypeptide
VLPNIQAVL

SEQ ID NO: 38 - protein, 9 aa, Artificial Sequence
  Synthetic Polypeptide
VEPSDTIEN

SEQ ID NO: 39 - protein, 9 aa, Artificial Sequence
  Synthetic Polypeptide
FFQSAIKLV

SEQ ID NO: 40 - protein, 9 aa, Artificial Sequence
  Synthetic Polypeptide
IQAVLLPKK

SEQ ID NO: 41 - protein, 9 aa, Artificial Sequence
  Synthetic Polypeptide
IAFSQYLQQ

SEQ ID NO: 42 - protein, 9 aa, Artificial Sequence
  Synthetic Polypeptide
FLGSFLYEY

SEQ ID NO: 43 - protein, 9 aa, Artificial Sequence
  Synthetic Polypeptide
FVNDIFERI

SEQ ID NO: 44 - protein, 9 aa, Artificial Sequence
  Synthetic Polypeptide
LPNIQAVLL

SEQ ID NO: 45 - protein, 9 aa, Artificial Sequence
  Synthetic Polypeptide
LLPGELAKH

SEQ ID NO: 46 - protein, 9 aa, Artificial Sequence
  Synthetic Polypeptide
FVAFVDKCC

SEQ ID NO: 47 - protein, 9 aa, Artificial Sequence
  Synthetic Polypeptide
LKPDPNTLC

SEQ ID NO: 48 - protein, 9 aa, Artificial Sequence
  Synthetic Polypeptide
MENFVAFVD

SEQ ID NO: 49 - protein, 9 aa, Artificial Sequence
  Synthetic Polypeptide
LFGDELCKV

SEQ ID NO: 50 - protein, 9 aa, Artificial Sequence
  Synthetic Polypeptide
VTIAQGGVL

SEQ ID NO: 51 - protein, 9 aa, Artificial Sequence
  Synthetic Polypeptide
MKSFVNDIF

SEQ ID NO: 52 - protein, 9 aa, Artificial Sequence
  Synthetic Polypeptide
LFTFHADIC

SEQ ID NO: 53 - protein, 9 aa, Artificial Sequence
  Synthetic Polypeptide
FVNDLFERL

SEQ ID NO: 54 - protein, 9 aa, Artificial Sequence
  Synthetic Polypeptide
IAQGGVLPN

SEQ ID NO: 55 - protein, 9 aa, Artificial Sequence
  Synthetic Polypeptide
LGSFLYEYS

SEQ ID NO: 56 - protein, 9 aa, Artificial Sequence
  Synthetic Polypeptide
FVDKCCAAD

SEQ ID NO: 57 - protein, 9 aa, Artificial Sequence
  Synthetic Polypeptide
LFEDTNLCA

SEQ ID NO: 58 - protein, 9 aa, Artificial Sequence
  Synthetic Polypeptide
VNFAEFSKK

SEQ ID NO: 59 - protein, 9 aa, Artificial Sequence
  Synthetic Polypeptide
MNSFVNDIF

SEQ ID NO: 60 - protein, 9 aa, Artificial Sequence
  Synthetic Polypeptide
MNSFVNDLF

SEQ ID NO: 61 - protein, 9 aa, Artificial Sequence
  Synthetic Polypeptide
LVDEPQNLI

SEQ ID NO: 62 - protein, 9 aa, Artificial Sequence
  Synthetic Polypeptide
MDVVYALKR

SEQ ID NO: 63 - protein, 9 aa, Artificial Sequence
  Synthetic Polypeptide
LLLPGELAK

SEQ ID NO: 64 - protein, 9 aa, Artificial Sequence
  Synthetic Polypeptide
LTPDETYVP

SEQ ID NO: 65 - protein, 9 aa, Artificial Sequence
  Synthetic Polypeptide
LQNEIDVSS

SEQ ID NO: 66 - protein, 9 aa, Artificial Sequence
  Synthetic Polypeptide
LVDFQDAKA

SEQ ID NO: 67 - protein, 9 aa, Artificial Sequence
  Synthetic Polypeptide
VGLFEDTNL

SEQ ID NO: 68 - protein, 9 aa, Artificial Sequence
  Synthetic Polypeptide
LGLIYEETR

SEQ ID NO: 69 - protein, 9 aa, Artificial Sequence
  Synthetic Polypeptide
ILGLIYEET

SEQ ID NO: 70 - protein, 9 aa, Artificial Sequence
  Synthetic Polypeptide
IDVSSREKS

SEQ ID NO: 71 - protein, 9 aa, Artificial Sequence
  Synthetic Polypeptide
LHTLFGDEL

SEQ ID NO: 72 - protein, 9 aa, Artificial Sequence
  Synthetic Polypeptide
LVGLFEDTN

SEQ ID NO: 73 - protein, 9 aa, Artificial Sequence
  Synthetic Polypeptide
IAQDFKTDL

SEQ ID NO: 74 - protein, 9 aa, Artificial Sequence
  Synthetic Polypeptide
FHADICTLP

SEQ ID NO: 75 - protein, 9 aa, Artificial Sequence
  Synthetic Polypeptide
FRIMAVLAI

SEQ ID NO: 76 - protein, 9 aa, Artificial Sequence
  Synthetic Polypeptide
IRIMAVLAS

SEQ ID NO: 77 - DNA, 1155 bp, Homo sapiens
ccggggggtcagggtcccagatgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagagtcactggaactggaggacccgtcttctgggctgggtgtgaccaagcaggatctgggcccagtccccatgtgagagcagcagaggcggtcttcaacatcctgccagccccacacagctacagctttcttgctcccttcagcccccagcccctcccccatctcccaccctgtacctcatcccatgagaccctggtgcctggctctttcgtcacccttggacaagacaaaccaagtcggaacagcagataacaatgcagcaaggccctgctgcccaatctccatctgtcaacaggggcgtgaggtcccaggaagtggccaaaagctagacagatccccgttcctgacatcacagcagcctccaacacaaggctccaagacctaggctcatggacgagatgggaaggcacagggagaagggataaccctacacccagaccccaggctggacatgctgactgtcctctcccctccagcctttggccttggcttttctagcctatttacctgcaggctgagccactctcttccctttccccagcatcactccccaaggaagagccaatgttttccacccataatcctttctgccgacccctagttccctctgctcagccaagcttgttatcagctttcagggccatggttcacattagaataaaaggtagtaattagaacaaaaaaaaaaaaaaaaaa

SEQ ID NO: 78 - RNA, 19 nt, Artificial Sequence
  Synthetic Polypeptide
gguaguaauuagaacaaaa

SEQ ID NO: 79 - RNA, 19 nt, Artificial Sequence
  Synthetic Polypeptide
gguucacauuagaauaaaa

SEQ ID NO: 80 - RNA, 19 nt, Artificial Sequence
  Synthetic Polypeptide
gaacaaaaaaaaaaaaaaa

SEQ ID NO: 81 - RNA, 19 nt, Artificial Sequence
  Synthetic Polypeptide
caaaaaaaaaaaaaaaaaa

SEQ ID NO: 82 - RNA, 19 nt, Artificial Sequence
  Synthetic Polypeptide
agaacaaaaaaaaaaaaaa

SEQ ID NO: 83 - RNA, 19 nt, Artificial Sequence
  Synthetic Polypeptide
acaaaaaaaaaaaaaaaaa

SEQ ID NO: 84 - RNA, 19 nt, Artificial Sequence
  Synthetic Polypeptide
guaauuagaacaaaaaaaa

SEQ ID NO: 85 - RNA, 19 nt, Artificial Sequence
  Synthetic Polypeptide
caugguucacauuagaaua

SEQ ID NO: 86 - RNA, 19 nt, Artificial Sequence
  Synthetic Polypeptide
guaguaauuagaacaaaaa

SEQ ID NO: 87 - RNA, 19 nt, Artificial Sequence
  Synthetic Polypeptide
ggcuuuucuagccuauuua

SEQ ID NO: 88 - protein, 295 aa, Homo sapiens
MHRRRSRSCREDQKPVMDDQRDLISNNEQLPMLGRRPGAPESKCSRGALYTGFSILVTLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM